(19) United States
(12) Patent Application Publication
(10) Pub. No.: US 2018/0010815 A1
(43) Pub. Date: Jan. 11, 2018

(54) System for monitoring fan filter units in clean rooms

(71) Applicant: Tsan-Chieh Wang, Tainan City (TW)

(72) Inventor: Tsan-Chieh Wang, Tainan City (TW)

(21) Appl. No.: 15/678,077

(22) Filed: Aug. 15, 2017

(30) Foreign Application Priority Data
May 5, 2017 (TW) 106206478

(51) Int. Cl. F24F 3/16; G01N 33/00; F24F 11/00

(52) U.S. Cl. CPC F24F 3/161; F24F 11/0086; G01N 33/0004

(57) ABSTRACT

A system for monitoring FFUs in each of clean rooms includes a first exhaust fan disposed above the clean rooms; lines each having one end connected to the FFUs in each clean room and the other end connected to a gateway; a personal computer communicating with the gateway; gas removal units disposed under the clean rooms and connected to a second exhaust fan; an emergency damper connected to the second exhaust fan; a CO₂ cylinders room disposed besides the emergency damper; and gas detectors, each communicating with the FFUs via the line, each gas detector having a connector connected to the gateway.

### BACKGROUND OF THE INVENTION

### 1. Field of the Invention

The invention relates to clean rooms and more particularly to a system for monitoring fan filter units in clean rooms.

### 2. Description of Related Art

A conventional system for monitoring fan filter units in clean rooms has many drawbacks including an independent network for monitoring, being difficult of maintaining and repairing, and poor adaptability.

Thus, the need for improvement still exists.

### SUMMARY OF THE INVENTION

It is therefore one object of the invention to provide a system for monitoring a plurality of fan filter units (FFUs) in each of a plurality of clean rooms, comprising a first exhaust fan disposed above the clean rooms; a plurality of lines each having one end connected to the FFUs in each clean room and the other end connected to a gateway; a personal computer communicating with the gateway; a plurality of gas removal units disposed under the clean rooms and connected to a second exhaust fan; an emergency damper connected to the second exhaust fan; a CO₂ cylinders room disposed besides the emergency damper; and a plurality of gas detectors, each communicating with the FFUs via the line, each gas detector including a connector configured to connect to the gateway for communicating with the personal computer.

The above and other objects, features and advantages of the invention will become apparent from the following detailed description taken with the accompanying drawings.

### BRIEF DESCRIPTION OF THE DRAWINGS

FIG. 1 schematically depicts a monitor system according to the invention;

FIG. 2 schematically depicts the FFUs; and

FIG. 3 schematically depicts an embodiment of the invention.

### DETAILED DESCRIPTION OF THE INVENTION

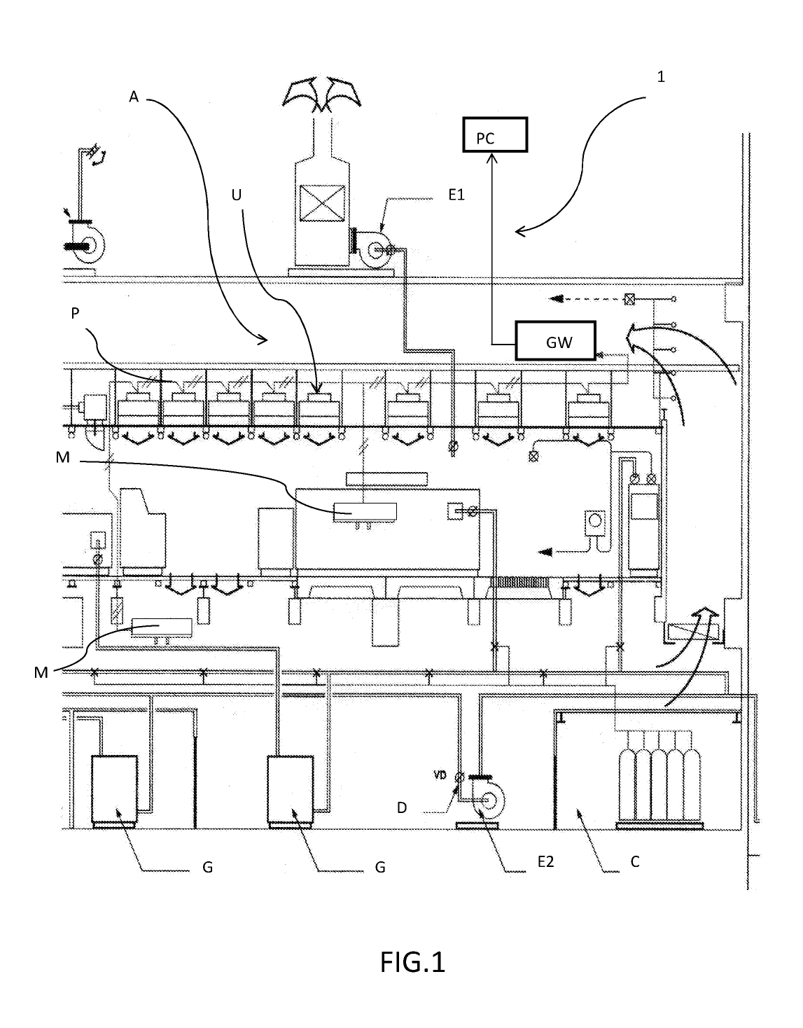

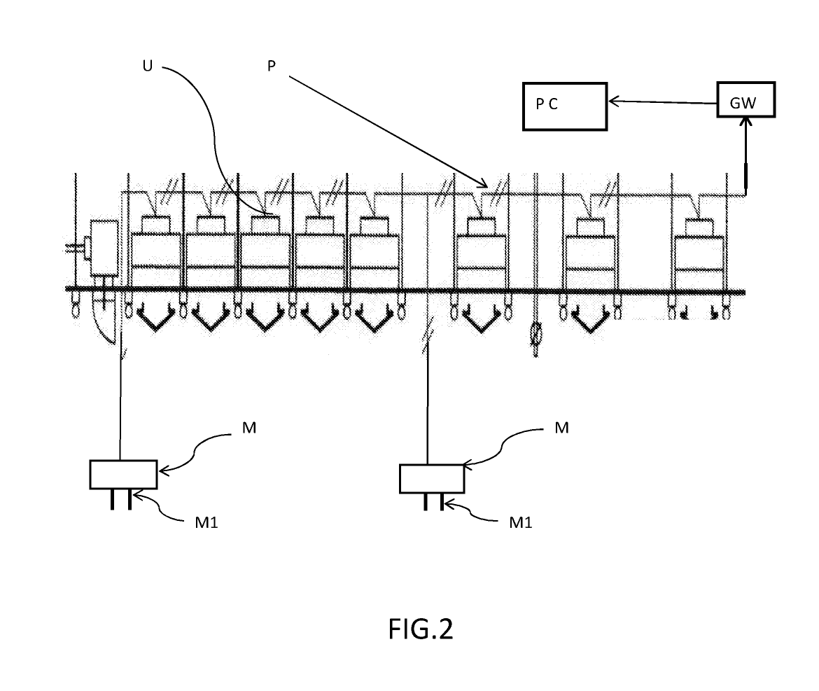

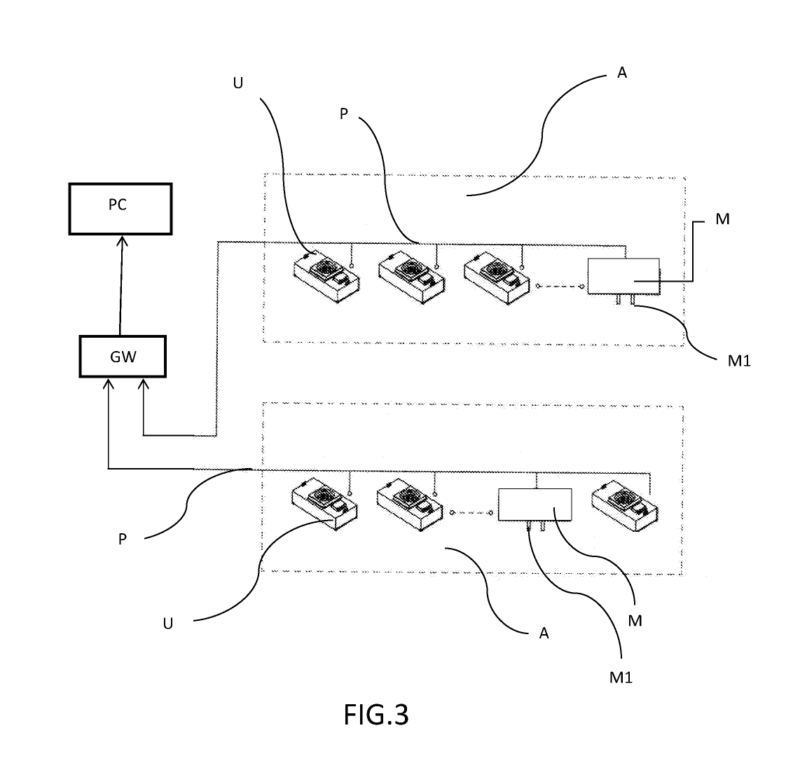

Referring to FIGS. 1 to 3, a monitor system 1 in accordance with the invention comprises a plurality of fan filter units (FFUs) U disposed in each of two clean rooms A; a first exhaust fan E1 disposed above the clean rooms A; two lines P each having one end connected to each FFU U in either clean room A or B, and the other end connected to a gateway GW; a personal computer PC communicating with the gateway GW; a plurality of gas removal units G disposed under the clean rooms A and connected to a second exhaust fan E2; an emergency damper D connected to the second exhaust fan E2; a CO₂ cylinders room C disposed besides the emergency damper D; and a plurality of gas detectors M each communicating with the FFUs U via the line P, the gas detector M including a connector M1 configured to connect to the gateway GW for communicating with the personal computer PC. A Human Machine Interface (HMI) is installed in the personal computer PC. The monitor system 1 is connected to MODBUS TCP/IP and MODBUS RTU.

While the invention has been described in terms of preferred embodiments, those skilled in the art will recognize that the invention can be practiced with modifications within the spirit and scope of the appended claims.

What is claimed is:

1. A system for monitoring a plurality of fan filter units (FFUs) in each of a plurality of clean rooms, comprising:
a first exhaust fan disposed above the clean rooms;
a plurality of lines each having one end connected to the FFUs in each clean room and the other end connected to a gateway;
a personal computer communicating with the gateway;
a plurality of gas removal units disposed under the clean rooms and connected to a second exhaust fan;
an emergency damper connected to the second exhaust fan;
a CO₂ cylinders room disposed besides the emergency damper; and
a plurality of gas detectors, each communicating with the FFUs via the line, each gas detector including a connector configured to connect to the gateway for communicating with the personal computer.
